(19) United States
(12) Patent Application Publication
(10) Pub. No.: US 2016/0287469 A1
(43) Pub. Date: Oct. 6, 2016

(54) EAR DEVICE

(71) Applicant: Ilich Vahimi, Seattle, WA (US)

(72) Inventor: Ilich Vahimi, Seattle, WA (US)

(21) Appl. No.: 15/085,043

(22) Filed: Mar. 30, 2016

Related U.S. Application Data
(60) Provisional application No. 62/140,269, filed on Mar. 30, 2015.

(51) Int. Cl. A61H 23/02; A61B 5/00

(52) U.S. Cl. CPC A61H 23/0263; A61B 5/0086; A61H 2201/0207; A61H 2201/5082

(57) ABSTRACT

A device configured for use in an ear canal of an ear is disclosed. The device includes a tip configured to be placed at the entrance of the ear canal, an electric vibrator coupled to the tip, a power source electrically coupled to the electric vibrator, a housing configured to enclose each of the electric vibrator and the power source, and a light based indicator configured to indicate at least one of a strength of the vibrations and a level of the power source. The vibrator is configured to generate vibrations. Further, the electric vibrator is coupled to the housing. The power source provides electrical energy for operation of the electric vibrator.

### FIELD OF THE INVENTION

The present invention pertains to an ear device. More particularly, the invention pertains to an ear device that assists in removing water lodged within the ear cavity and relieving built up pressure within the ear cavity.

### BACKGROUND OF THE INVENTION

A common problem experienced after swimming, diving, or showering is trapped water within the ear canal. Removal of water trapped within the ear usually involves waiting for gravity to relieve the pressure of the trapped water, shaking the head vigorously for a certain amount of time, or attempting to remove the water immediately by inserting a finger, q-tip, or similar pointed object into the ear canal. If the preferred option is waiting for gravity, water may be stuck within the ear for hours or even days. If the preferred option is to shake the head, a lot of effort and energy is required to relieve the pressure from within the ear. If the preferred option is to insert a finger, q-tip, or pointed object, there is an increased risk of introducing infection into the ear canal even if the removal of water is successful or not. If left untreated, the water lodged within the ear may result in an even more serious infection and long-term damages to the ear.

Accordingly, there is a need for an improved solution, which effectively, easily, and safely relieves the ear from water lodged within the ear canal and built up pressure from diving and flying.

### SUMMARY OF THE INVENTION

The present disclosure provides a device configured for use in an ear canal of an ear. The device includes a tip configured to be placed at the entrance of the ear canal, an electric vibrator coupled to the tip, a power source electrically coupled to the electric vibrator, a housing configured to enclose each of the electric vibrator and the power source, and a light based indicator configured to indicate at least one of a strength of the vibrations and a level of the power source. The electric vibrator is configured to generate vibrations. Further, the electric vibrator is coupled to the housing. The power source provides electrical energy for operation of the electric vibrator.

In one aspect of the present disclosure, the device is a hand-held device.

In another aspect of the present disclosure, the device is a wearable device.

The device provides an easy, effective, and safe way for removing water lodged within the ear cavity and built up pressure in the ear cavity.

### BRIEF DESCRIPTION OF THE DRAWINGS

FIG. 1 is a perspective view of an ear device according to an embodiment of the present disclosure;

FIG. 2 is a right side elevational view thereof;

FIG. 3 is a front elevational view thereof;

FIG. 4 is a top view thereof;

FIG. 5 is an exploded view of the ear device of FIG. 1, according to some embodiments of the present disclosure.

FIG. 6A is a perspective view of a tip of the ear device of FIG. 1;

FIG. 6B is a side view of the tip of FIG. 6A;

FIG. 7A is a perspective view of a bud of the ear device of FIG. 1;

FIG. 7B is a side view of the bud of FIG. 7A;

FIG. 8 is a back elevational view of the ear device of FIG. 1;

FIG. 9 is a top view of an ear device according to another embodiment of the present disclosure.

### DETAIL DESCRIPTION OF THE INVENTION

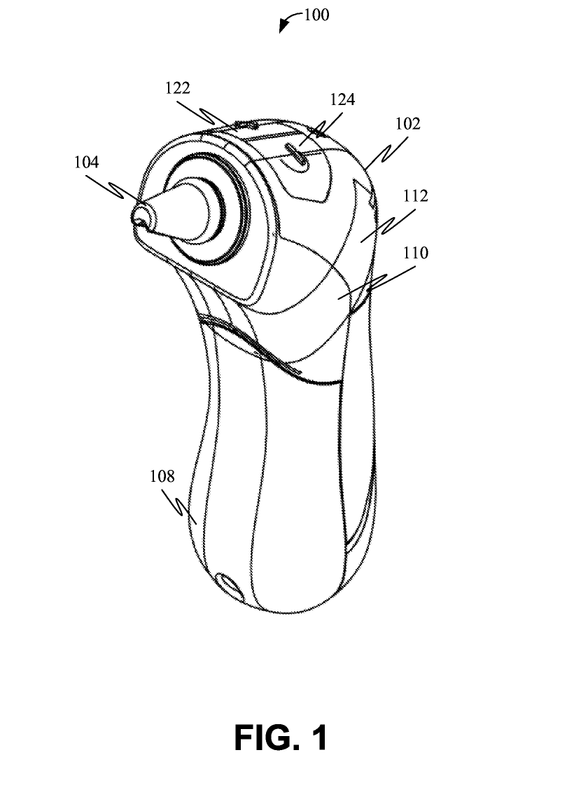

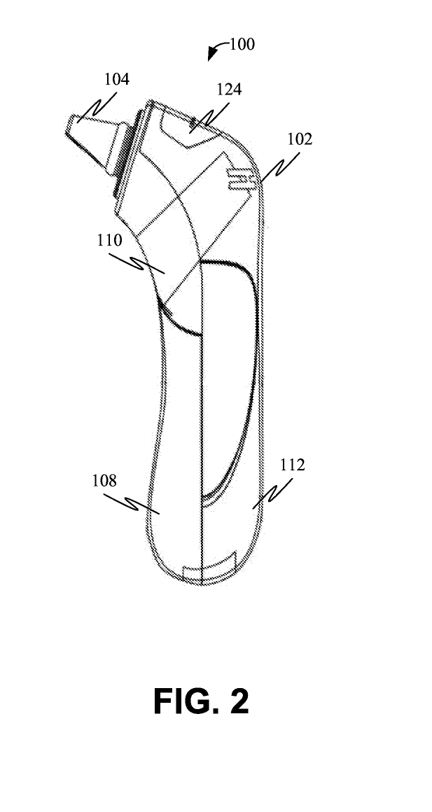

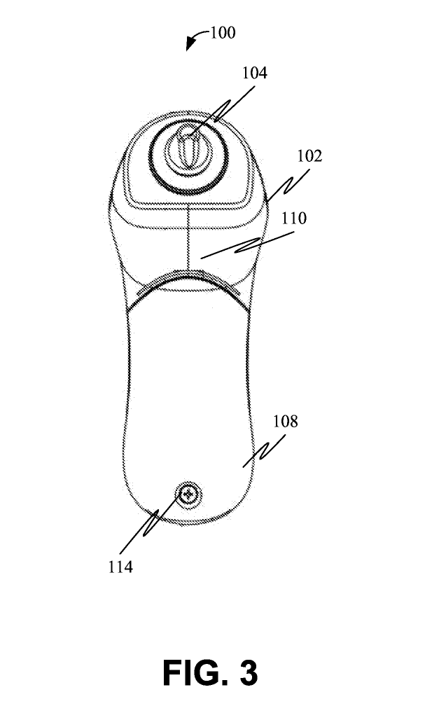

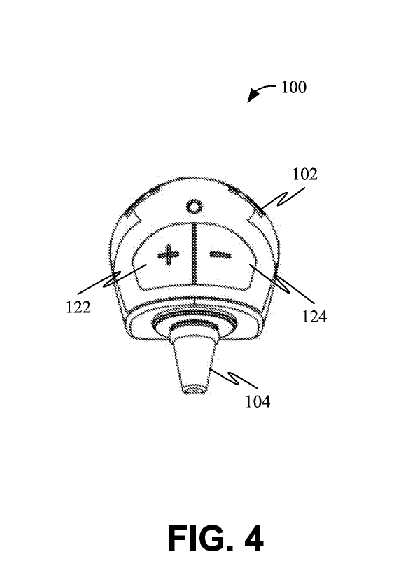

All illustrations of the drawings are for the purpose of describing selected versions of the present invention and are not intended to limit the scope of the present invention. In this regard, it should be noted that the drawings, particularly FIG. 1 through FIG. 5, may not show all of the components of the present invention to the scales or in the shapes that may be utilized in physically constructing the present invention.

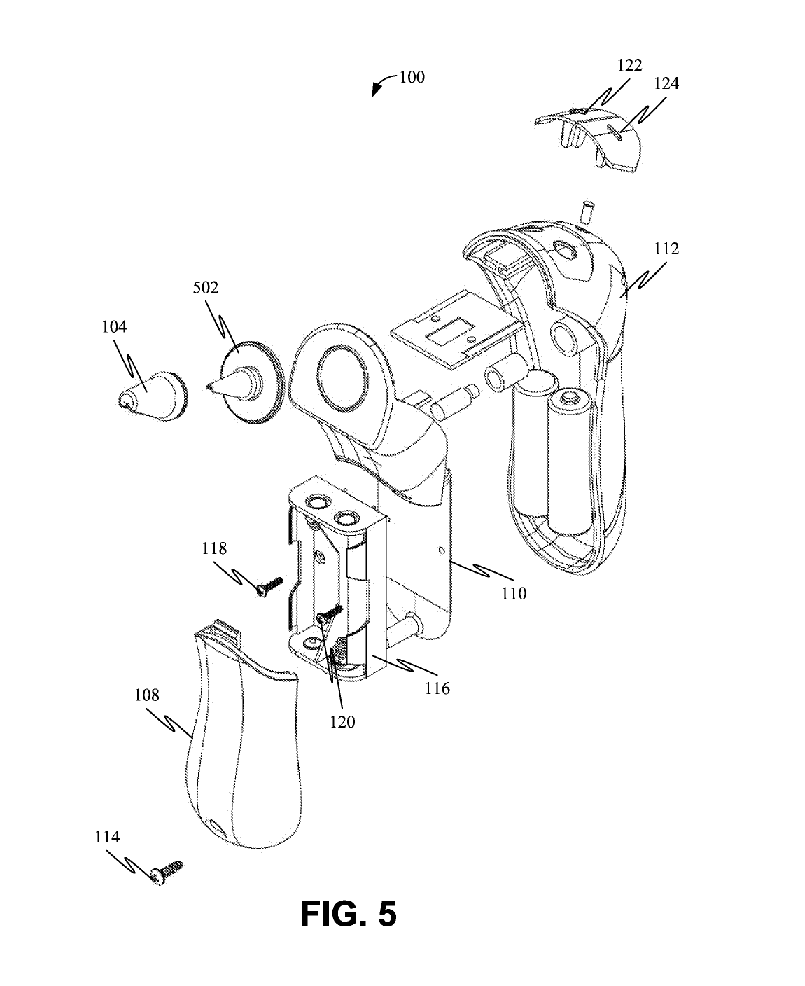

As further illustrated in FIGS. 1-5, an ear device 100 has an elongated housing 102 and a conical ear tip 104 adapted to be placed in the ear canal of a person. The ear tip 104 may be transversely connected to a top end of the housing 102. In a further embodiment (shown in FIG. 5), the housing 102 may include a front panel 108, a middle panel 110 and a back panel 112, which are joined together to create a hollow space within the housing 102. The front panel 108, the middle panel 110 and the back panel 112 may be joined together using one or more screws (for example, a screw 114). The housing 102 may be ergonomically shaped to be comfortably held in hand of a user. The housing 102 may also be configured to be water resistant.

Further, the housing 102 may be configured to enclose one or more components. For example, the housing 102 may enclose an electric vibrator (not shown), which is configured to generate vibrations. The electric vibrator may be one of an eccentric rotating mass motor and a linear resonant actuator. The electric vibrator may be coupled to the housing 102 and the tip 104. The vibrations generated by the electric vibrator travel via the tip 104 into the ear canal and relieve the pressure and the water lodged within the ear. Further, the vibrations generated by the electric vibrator may provide soothing experience in the ear canal. The housing 102 may also include a door (for example, the front panel 108) configured to provide physical access to the electric vibrator.

Yet further, the housing 102 may enclose a power source (not shown). The power source may be electrically coupled to the electric vibrator, wherein the power source provides electrical energy for operation of the electric vibrator. The power source may be a replaceable battery placed inside a battery holder 116, which may be screwed to the middle panel 110 using screws 118-120. The housing 102 may also include a door (for example, the front panel 108) configured to provide physical access to the power source.

Moreover, the housing 102 may include a light based indicator (not shown) configured to indicate one or both of strength of the vibrations generated the electric vibrator and a status of the power source. The light generated by the light based indicator may be visible from outside the housing 102. Light based indicator may include one or more LEDs.

The ear device 100 further includes a controller electrically coupled to one or both of the power source and the electric vibrator, wherein the controller is configured to control the strength of the vibrations. A user may provide inputs to the controller using a control panel. For example, the control panel may include an “up” button 122 and a “down” button 124. A user may use the “up” button 122 and the “down” button 124 to control the strength of the vibrations generated the electric vibrator. By pressing the “up” button 122, the user may increase the strength of vibrations. Similarly, by pressing the “down” button 124, the user may decrease the strength of vibrations.

In a further embodiment, the housing 102 further includes a fan (not shown) electrically coupled to the power source. The fan is configured to generate an airflow, wherein the tip 104 includes a pinhole to channel the airflow from the fan into the ear canal of a user. When the tip 104 is placed in the ear canal of a person, then the fan may be switched-on to help evaporate the water in the ear canal.

In another further embodiment, the housing 102 includes a heater configured to generate heat. The tip 104 is configured to transmit heat generated by the heater into the ear canal. The heat may be transferred to the ear canal via conduction from the body of the tip 104 to the ear canal. The heat may also be transferred to the ear canal via convection along with the air flow generated by the fan and/or via radiation. The generated heat may also assist in evaporating the water in the ear canal. Further, the housing 102 may include a temperature sensor configured to sense body temperature through the ear canal. Still further, the housing 102 may include a wireless transceiver communicatively coupled to one or both of the heater and the temperature sensor. The wireless transceiver is configured to communicate temperature data with an external device (such as a smartphone). The temperature data may include one or both of a temperature sensed by the temperature sensor and a temperature of heat to be generated by the heater. The wireless transceiver may employ any suitable communication protocol, for example BlueTooth™ and Wi-Fi, to communicate with the external device. A software application installed on the external device may be used to manage communications with the wireless transceiver of the ear device 100. The software application may synchronize data stored in the external device with the data from the ear device 100. Further, the software application may be used to control various components of the ear device 100; for example, switching-on the fan, and controlling the fan speed, the strength of the vibrations, or the heat generated by the heater. The fan and the heater may also help in removing earwax or cerumen from the ear canal.

In another embodiment, the housing 102 may include an audio speaker configured to generate sound waves. Alternatively, the electric vibrator may be further configured to generate sound waves. Further, the housing 102 may include a wireless receiver communicatively coupled to the audio speaker, wherein the wireless receiver is configured to wirelessly receive music data from an external device (such as a smartphone). The audio speaker is configured to reproduce sound based on the music data received from the external device. Further, the housing 102 may include a memory to store music, which is then played using the audio speaker. The audio speaker may automatically play music when the tip 104 is inserted into the ear canal of the user. The music may help calm the user while the ear device 100 is being used.

The ear device 100 may include multiple control panels located at various places on the outer cover of the housing. The control panels may be used to control operation of one or more of a fan, a heater, a temperature sensor, a wireless transceiver and an audio speaker. Further, the control panels may include audio control buttons such as a status display, a play button, a pause button, and program selection buttons.

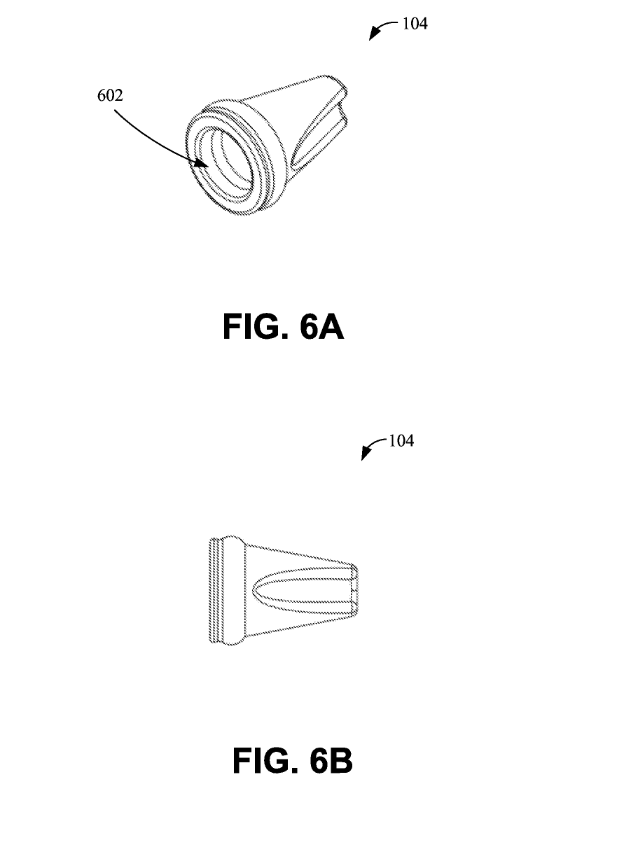

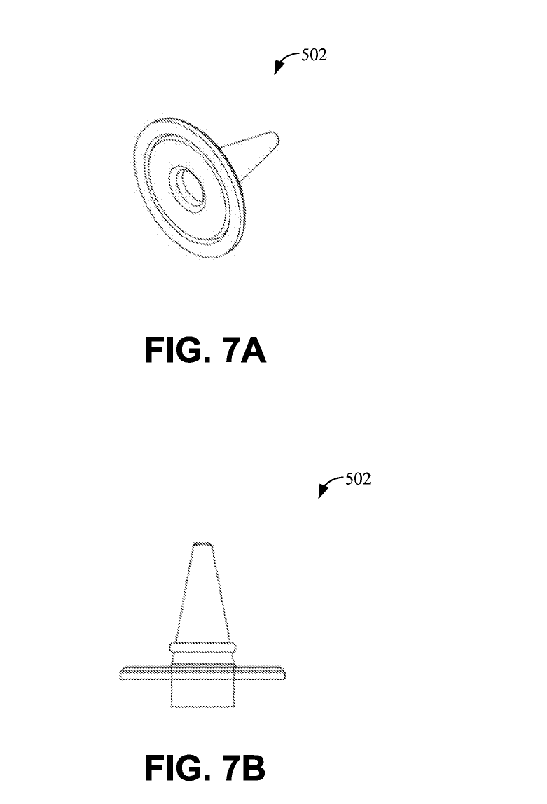

As shown in FIGS. 6A and 6B, the tip 104 is a conical-shaped so that it can be easily inserted inside the ear canal. The tip 104 is connected to the electric vibrator via a bud 502. The bud 502 is shown in FIGS. 5, 7A and 7B. The bud 502 is significantly smaller than the tip 104, so that the tip 104 may surround the bud 502. The bud 502 serves as the base on which the tip 104 is secured to the housing 102. The tip 104 is a padded member that comprises a receiving end 602. The receiving end 602 is a depression within the member that comprises the necessary structure to receive the bud 502. A user may replace the tip 104 when required. Further, the user may be allowed to select best-fit tip from a plurality of available tips with a variety of ergonomic shapes. The tip 104 may include a waterproof material that may be sanitized for use by a plurality of users. Alternatively, the tip 104 may comprise materials that allow for one-time use for sanitary reasons.

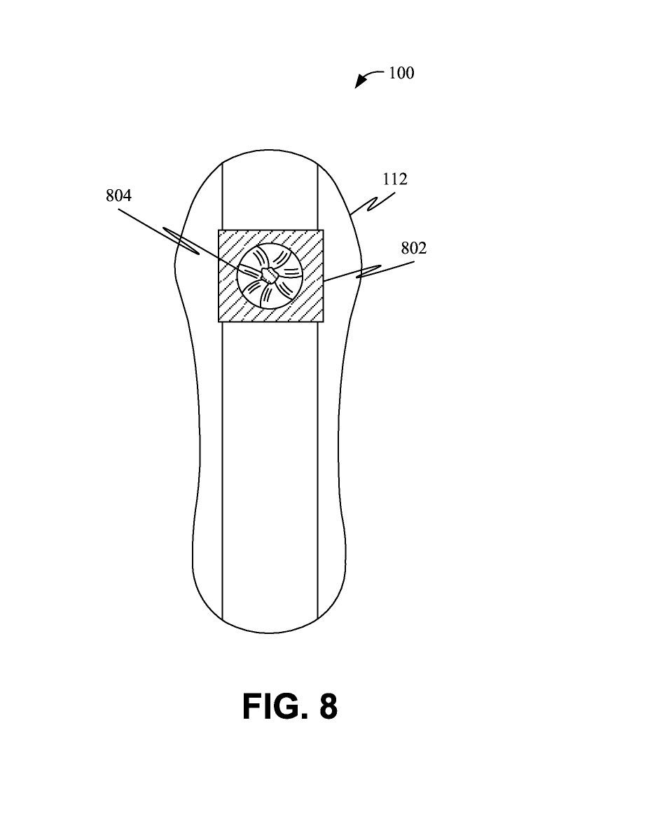

The housing 102 may also include a camera (not shown) that is configured to click photos or shoot videos of the inside view of the ear canal of the user via the pinhole in the tip 104. As shown in FIG. 8, the housing 102 may include a small display 802 on the back panel 112 to show an inside view 804 of the ear canal (captured by the camera) while the tip 104 is inserted in the ear canal. This may help the user determine the level of evaporation required. The camera may be an infrared camera. Further, the camera may provide high image quality, enhanced sensitivity, and high live frame rates at full resolution to enable microscopic inspection of the ear canal.

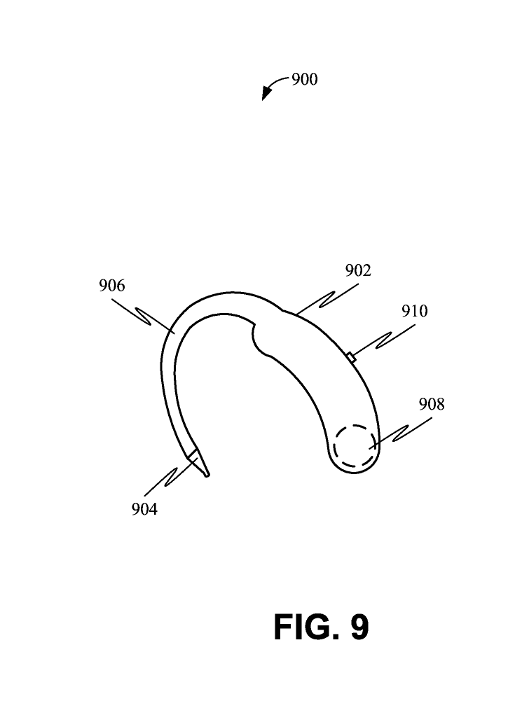

In an alternate embodiment, the ear device may be wearable ear device 900 as shown in FIG. 9. The wearable ear device 900 includes a housing 902 and a conical ear tip 904 adapted to be placed in the ear canal of a person. The housing 902 comprises an ergonomic shape that allows the housing 902 to be comfortably engaged with the back of the helix of an ear and the scalp.

The wearable ear device 900 further includes a stabilizing rod 906 that connects the housing 902 to the tip 904. The stabilizing rod 906 is an ergonomic, bendable rod-like member that contours from the portion of the ear behind the upper helix, around the root of the ear along the root of the helix, and into the external auditory mestus of the ear. Various components of the wearable ear device 900 may be made using waterproof materials. Further, the materials may have the necessary durability and flexibility.

The housing 902 is a hollow casing that encloses various components including a power source, and an electric vibrator. The housing 902 comprises waterproof materials that protect the electrical connectivity, the power source, and the electric vibrator. The power source may be a removable battery, such as a button cell battery 908. The power source may be secured within the housing 902 via a door similar to that of similar small electronics powered by button cell batteries. The door may be secured to the housing 902 via a screw or a snap mechanism.

The electric vibrator may be an actuator powered by the power source and controlled by a power switch. The actuators may be eccentric rotating mass motors or linear resonant actuators. The electric vibrator creates vibrations throughout the housing 902 that travels through the stabilizing rod 906 to the tip 904. For example, the electric vibrator includes the necessary components similar to that of vibration mechanisms of cellular phones. The power switch may include the necessary electrical components to connect to the power source and a knob 910. The knob 910 protrudes out of the housing 902 so that a user may engage the knob 910 to control the electric vibrator. The knob 910 in its engaged position allows the power source to activate the electric vibrator. The knob 910 in its retracted position may prevent the power source from activating the electric vibrator. Alternate embodiments of the power switch may comprise a button mechanism, a dial mechanism, or any other similar power controlling mechanism. Further, the power switch may be able to control the strength of the vibrations of the electric vibrator.

The stabilizing rod 906 is attached to a platform of an extrusion of the housing 902, providing a path for the vibrations created by the electric vibrator to travel to the tip 904. The stabilizing rod 906 comprises silicone-like materials for a flexible and durable connection between the housing 902 and the tip 904. The stabilizing rod 906 comprises a first end and a second end. The first end and second end are opposite each other. The first end comprises an attaching member that links to the platform of the extrusion of the housing 902 that corresponds to the stabilizing rod 906. The attaching member may be a female or male member depending on the orientation of the platform. The second end comprises a bud that is meant to secure the tip 904 to the stabilizing rod 906. The bud is significantly smaller than that of the tip 904 so that the tip 904 may surround the bud of the stabilizing rod 906. The bud serves as the base for which the tip 904 is secured. A user may replace the tip 904 when required. Further, the extrusion corresponding to the stabilizing rod 906 may allow the removal and insertion of the stabilizing rod 906 at the discretion of the user.

The tip 904 is a padded member that comprises a receiving end. The receiving end is a depression within the member that comprises the necessary structure to receive the bud of the second end of the stabilizing rod 906. The tip 904 engages with the ear canal and receives the vibrations of the electric vibrator within the housing 902 via the stabilizing rod 906. Users may be allowed to select best-fit tip from a plurality of tips with a variety of ergonomic shapes. The tip 904 may include a waterproof material that may be sanitized for a plurality of users. Alternatively, the tip 904 may comprise materials that allow for one-time use for sanitary reasons.

A user may place the tip 904 along the edge of the ear canal. Once securely and comfortably positioned, the housing 902 may be pulled back behind the ear. The housing 902 rests between the helix of the ear and his or her hear. The stabilizing rod 906 comfortably curves and adjusts to the unique contours of the user's ear. The stabilizing rod 906 extends past the front of the ear and curves around the root of the helix. The wearable ear device 900 is fully engaged with the ear and may be left on the ear without any assistance by the user. At the user's discretion, the user may engage the power switch of the housing 902 to activate the vibration mechanism. The tip 904 vibrates against the ear canal, dislodging the water trapped within due to pressure. The vibration mechanism may be left on until the user feels that all the water is released from the ear canal. Once the removal of water is complete, the user may disengage the power switch and the vibration mechanism stops delivering vibrations to the tip 904. The wearable ear device 900 may be removed and the ear may be wiped to remove any leakage from the ear canal. The tip 904 may be removed and replace with another tip that comprises a varying external design to better accommodate user's ear.

The ear device of the present disclosure helps in safely, quickly, and easily removing water lodged within the ear canal of a user.

Although the invention has been explained in relation to its preferred embodiment and alternative embodiment, it is to be understood that many other possible modifications and variations can be made without departing from the spirit and scope of the invention.

The following is claimed:

1. A device configured for use in an ear canal of an ear, the device comprising:
a. a tip configured to be placed at the entrance of the ear canal;
b. an electric vibrator coupled to the tip, wherein the vibrator is configured to generate vibrations;
c. a power source electrically coupled to the electric vibrator, wherein the power source provides electrical energy for operation of the electric vibrator;
d. a housing configured to enclose each of the electric vibrator and the power source, wherein the electric vibrator is coupled to the housing; and
e. a light based indicator configured to indicate at least one of a strength of the vibrations and a level of the power source.

2. The device of claim 1 further comprising means to transmit vibrations from the electric vibrator to the tip.

3. The device of claim 1, wherein the housing is configured to be held in a hand.

4. The device of claim 3, wherein the housing is ergonomically shaped to be comfortably held in hand.

5. The device of claim 1, wherein the housing is water resistant.

6. The device of claim 1, wherein the power source comprises a battery.

7. The device of claim 1, wherein the housing comprises a door configured to provide physical access to the power source.

8. The device of claim 1, wherein the housing comprises a door configured to provide physical access to the electric vibrator.

9. The device of claim 1, wherein the electric vibrator comprises at least one of an eccentric rotating mass motor and a linear resonant actuator.

10. The device of claim 1 further comprising a controller electrically coupled to at least one of the power source and the electric vibrator, wherein the controller is configured to control the strength of the vibrations.

11. The device of claim 1, wherein the electric vibrator coupled to the tip via a bud, wherein the bud configured to be surrounded by the tip.

12. The device of claim 11, wherein the tip is removably attached to the bud.

13. The device of claim 1 further comprising a fan electrically coupled to the power source, wherein the fan is configured to generate an airflow, wherein the tip is further configured to channel the airflow into the ear canal.

14. The device of claim 1 further comprising a heater configured to generate heat, wherein the tip is further configured to transmit heat into the ear canal.

15. The device of claim 14 further comprising a temperature sensor configured to sense body temperature through the ear canal.

16. The device of claim 1 further comprising an audio speaker configured to generate sound waves.

17. The device of claim 1, wherein the electric vibrator is further configured to generate sound waves.

18. The device of claim 16 further comprising a wireless receiver communicatively coupled to the audio speaker, wherein the wireless receiver is configured to wirelessly receive music data from an external device, wherein the audio speaker is configured to reproduce sound based on the music data.

19. The device of claim 15 further comprising a wireless transceiver communicatively coupled to at least one of the heater and the temperature sensor, wherein the wireless transceiver is configured to communicate temperature data with an external device, wherein the temperature data comprises at least one of a temperature sensed by the temperature sensor and a temperature of heat to be generated by the heater.

20. The device of claim 1 further comprising a camera electrically coupled to the power source, wherein the camera is configured to capture a view of an inside of the ear canal.

21. The device of claim 20 wherein the camera is an infrared camera.

22. The device of claim 20 wherein the camera provides image quality required for microscopic inspection of the ear canal.
